(19) United States
(12) Patent Application Publication
(10) Pub. No.: US 2009/0005789 A1
(43) Pub. Date: Jan. 1, 2009

(54) Force Sensitive Foot Controller

(76) Inventor: Steven T. Charles, Memphis, TN (US)

Correspondence Address: ALCON, IP LEGAL, TB4-8, 6201 SOUTH FREEWAY, FORT WORTH, TX, 76134, US

(21) Appl. No.: 11/768,568

(22) Filed: Jun. 26, 2007

(51) Int. Cl. A61B 19/00

(52) U.S. Cl. 606/130

(57) ABSTRACT

A microsurgical system, and a foot controller for the improved operation of a microsurgical system, are disclosed. A surgeon may use the foot controller to proportionally control a surgical parameter based upon the amount of force applied to a pressure plate of the foot controller.

### FIELD OF THE INVENTION

The present invention generally pertains to surgical systems. More particularly, but not by way of limitation, the present invention pertains to foot controllers for the operation of such systems.

### DESCRIPTION OF THE RELATED ART

Various foot controllers are used to control surgical systems, and particularly ophthalmic microsurgical systems. During ophthalmic surgery, a surgeon views the patient's eye through an operating microscope. To control the microsurgical system and its associated handpieces during the various portions of the surgical procedure, the surgeon must either instruct a nurse how to alter the machine settings on the surgical system, or use the foot controller to change such settings. Where possible, many surgeons prefer to use the foot controller to alter the machine parameters or settings on the surgical system, eliminating the need to converse with a nurse during the surgical procedure. Most prior art foot controllers have a hinged upper surface which is movable through a range of motion. An encoder within the foot controller is able to detect the exact position of the upper surface and provide either linear or non-linear proportional control of surgical parameters. However, the use of such foot controllers requires the surgeon moving the foot pedal through a possibly wide range of motion, and can prove uncomfortable for a surgeon to use, particularly during extended surgical procedures. In addition, such a foot controller would not be desirable if the surgeon has limited ankle mobility. Finally, such position sensing foot controllers are expensive to manufacture, and can be quite large and heavy.

Therefore, a need continues to exist for an improved foot controller, for use with powered microsurgical devices, that is compact, inexpensive to manufacture, and can be comfortably and reliably controlled by the surgeon.

### SUMMARY OF THE INVENTION

In a preferred embodiment, the present invention comprises a method of providing proportional control of a parameter in a microsurgical system. The microsurgical system has a computer, a foot controller operatively coupled to the computer, and a surgical parameter. The foot controller has a pressure plate and a force sensor coupled to the pressure plate. An amount of force applied to the pressure plate is determined. A value of the surgical parameter is proportionally controlled as a function of the force applied to the pressure plate.

In another embodiment, the present invention comprises a microsurgical system having a computer, a foot controller operatively coupled to the computer, and a surgical parameter. The foot controller has a pressure plate and a force sensor coupled to the pressure plate.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a schematic view of a microsurgical system according to a preferred embodiment of the present invention.

FIG. 2 is a top perspective view of a foot controller of a microsurgical system according to a preferred embodiment of the present invention.

FIG. 3 is a cross sectional view of a foot controller of a microsurgical system according to a preferred embodiment of the present invention.

### DETAILED DESCRIPTION OF THE PREFERRED EMBODIMENTS

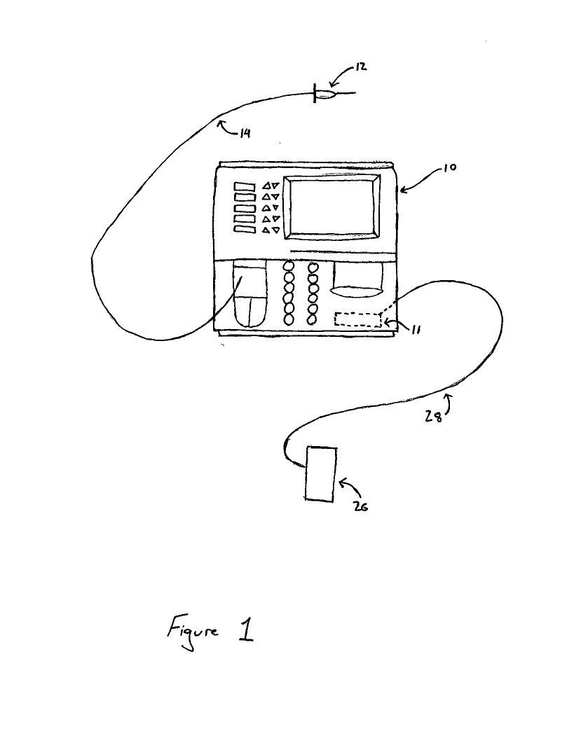

The preferred embodiments of the present invention and their advantages are best understood by referring to FIGS. 1 through 3 of the drawings, like numerals being used for like and corresponding parts of the various drawings.

FIG. 1 shows a microsurgical system 10 according to a preferred embodiment of the present invention. As shown in FIG. 1, microsurgical system 10 is an ophthalmic microsurgical system. However, microsurgical system 10 may be any microsurgical system, including a system for performing otic, nasal, throat, or other surgeries. System 10 has computer 11 disposed therein. System 10 is capable of providing ultrasound power, pneumatic drive pressure, irrigation fluid, and aspiration vacuum to microsurgical instrument 12. Instrument 12 may be any microsurgical instrument necessary for performing otic, nasal, throat, or other surgeries, but is most preferably an instrument to be used in either anterior or posterior segment ophthalmic microsurgery, such as a laser, diathermy probe, a phacoemulsifier, vitreous cutter, powered scissors, powered forceps, or powered fluid injector. Surgical instrument 12 is operatively coupled to system 10 via interface 14. A foot controller 26 is operatively connected to surgical system 10 via interface 28. Interfaces 14 and 28 may be conventional electronic cable or standard surgical tubing as dictated by the requirements of instrument 12.

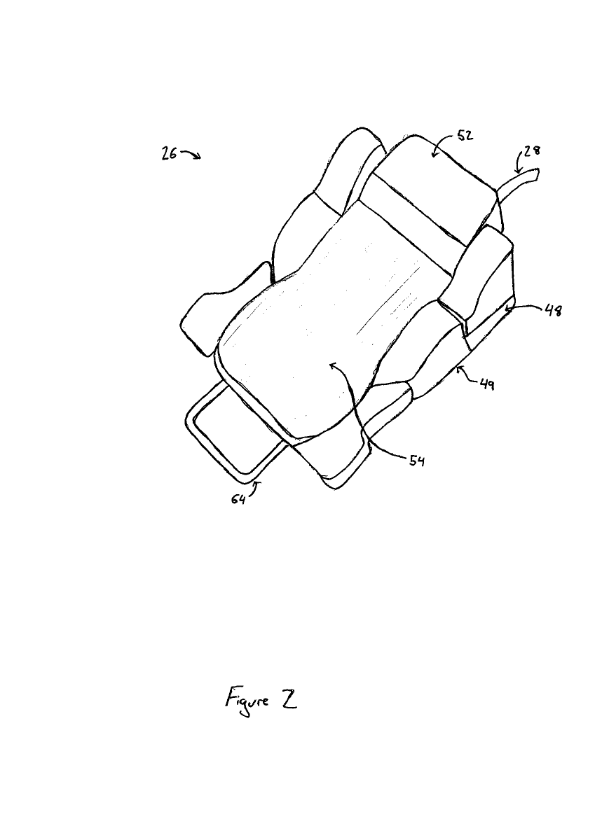

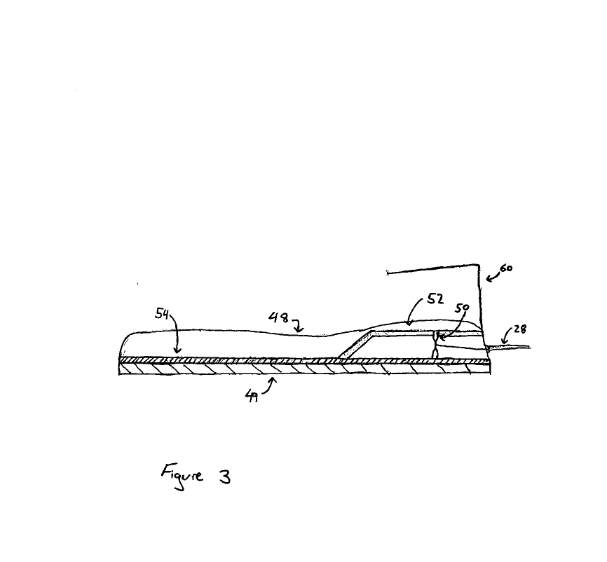

FIGS. 2 and 3 show a preferred embodiment of foot controller 26. Foot controller 26 has a body 48 with a base 49 that supports foot controller 26 on the operating room floor. Body 48 preferably includes a pressure plate 52, heel rest 54, and a handle 64. Force sensor 50 is disposed within the body and mechanically coupled to pressure plate 52. Pressure plate 52 may be made of any appropriate material but is most preferably made of a lightweight inexpensive material, such as aluminum. Force sensor 50 may be any appropriate device, but is most preferably a strain gauge or a force sensing resistor such as the FLEXIFORCE® force sensing resistor available from Interlink Electronics or Tekscan, Inc. of Boston, Mass. Force sensor 50 is electrically coupled to interface 28. Interface 28 is, in turn, electrically coupled to system 10. As shown in FIG. 3, shield 60 may be coupled to body 48 to partially enclose pressure plate 52. Shield 60 serves to protect foot controller 26 from accidental actuation.

During operation, the surgeon proportionally controls a surgical parameter related to the operation of a surgical handpiece by applying pressure to pressure plate 52. Such pressure slightly deforms force sensor 50 causing an electric signal to be generated that is proportional to the applied pressure. This electric signal is transmitted to surgical system 10 via interface 28. Computer 11 of system 10 then proportionally controls a surgical parameter of instrument 12 as a function of the amount of force applied to pressure plate 52. Such proportional control may be in either a linear or non-linear fashion.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A method of providing proportional control of surgical instruments in a microsurgical system comprising the steps of:
providing a microsurgical system, said system comprising:
a computer;
a foot controller operatively coupled to said computer, said foot controller having a force sensor coupled to a pressure plate; and
at least one surgical parameter;
determining an amount of force applied to said pressure plate; and
proportionally controlling a value of at least one of said surgical parameters as a function of said force applied to said pressure plate.

2. The method of claim 1 wherein said step of proportionally controlling said value of said surgical parameter comprises varying said value of said surgical parameter with said force applied to said pressure plate in a linear manner.

3. The method of claim 1 wherein said step of proportionally controlling said value of said surgical parameter comprises varying said value of said surgical parameter with said force applied to said pressure plate in a non-linear manner.

4. The method of claim 1 wherein said force sensor is a strain gauge.

5. The method of claim 1 wherein said force sensor is a force sensing resistor.

6. The method of claim 1 wherein said microsurgical system further comprises a shield coupled to said foot controller, said shield at least partially enclosing said pressure plate.

7. The method of claim 1 wherein:
said microsurgical system comprises a surgical handpiece; and
said surgical parameter is associated with operation of said handpiece.

8. A microsurgical system comprising:
a computer;
a foot controller operatively coupled to said computer, said foot controller having a force sensor coupled to a pressure plate; and
at least one surgical parameter.

9. The system of claim 8 wherein said force sensor is a strain gauge.

10. The system of claim 8 wherein said force sensor is a force sensing resistor.

11. The system of claim 8 further comprising a shield connected to said foot controller, said shield at least partially surrounding said pressure plate.

12. The system of claim 8 further comprising a microsurgical handpiece, and wherein said surgical parameter is associated with operation of said handpiece.
